(19) United States
(12) Patent Application Publication
(10) Pub. No.: US 2012/0123526 A1
(43) Pub. Date: May 17, 2012

(54) THORACIC AORTIC STENT STRUCTURE

(76) Inventors: Po-Jen Ko, Taipei City (TW); Ching-Yang Wu, Taipei County (TW); Yun-Hen Liu, Taoyuan County (TW)

(21) Appl. No.: 12/944,152

(22) Filed: Nov. 11, 2010

(51) Int. Cl. A61F 2/82

(52) U.S. Cl. 623/1.35

(57) ABSTRACT

A thoracic aortic stent structure, including a main stent having at least one sub-stent which is formed as a piece on the main stent; and a membrane covering the main stent and the at least one sub-stent, and having at least one side-opening corresponding to a free end of the at least one sub-stent. The membrane compresses the main stent and the at least one sub-stent, which causes diameter of the main stent and the at least one sub-stent narrower. Upon removing the membrane, the main stent and the at least one sub-stent can extend to reconstruct a vascular pathway. The thoracic aortic stent is able to simplify the process, of placement, save time and provide easy operation.

### FIELD OF THE INVENTION

This invention relates to a thoracic aortic stent structure, and more particularly to a thoracic aortic stent structure which is able to simplify the process of placement, save time and provide easy operation.

### BACKGROUND OF THE INVENTION

Thoracic abdominal aortic aneurysm usually results from atherosclerosis or infection which weakens aortic wall and the aortic is gradually swollen under high blood pressure for a long time. Clinically, if the diameter of the aortic aneurysm is larger than 5 centimeter or a patient continues to feel chest pain, it is necessary for the patient to receive surgery.

Conventionally, the surgery is an open surgery and thoracotomy from left posterior and lateral and using vascular clamp to clamp distal and proximal arteries of the thoracic abdominal aortic aneurysm. The thoracic abdominal aortic aneurysm is then being cut after the blood flow is under control, and a pathway of the thoracic, abdominal aorta is reconstructed by connecting an artificial artery with the rest of the original artery. In addition, each visceral aortic vessels originated from the thoracic abdominal aorta has to be connected to the artificial artery with new blood pathway. However, traditional surgery takes long time, the patient loses large amount of blood during operation, and postoperative pulmonary atelectasis causes lung complications.

The development of stents began since the late '90s to reconstruct the normal vascular flow path by inserting the stents to separate the aneurysm. It is so called the endovascular aneurysm repair (EVAR). In clinical endovascular aneurysm repair practice, a metal wire is inserted from both sides of the femoral artery, and an appropriate stent size would be determined by surgery angiography according to the location, length and influential area of the aneurysm. The stent is then guided to an expected destination through the metal wire. After the membrane wrapped outside the stent is removed, the stent can fully extend to reconstruct vascular flow path. According to the clinical study, the endovascular aneurysm, comparing with the conventional surgery, has the following advantages of (1) shorter operative time, (2) small amount of blood loss, and (3) shorter recovery time.

Referring to FIGS. 5A to 5E, the conventional steps of setting up a thoracic aortaic stent includes A: sending a main stent 42 covered with a membrane 41 through a metal wire 40 from the lateral femoral arteries of both sides to the position of thoracic aortic arch with the assistance of X-ray to the main stent 42 in the vessel; B: removing the membrane 41 so that the main stent 42 can fully extend in the thoracic aortic arch until the blood flows into the rain stent 42; C: after the main stent 42 is secured, the metal wire 40 penetrates a preserved hole 43 on the main stent and a sub-stent 44 is moved to the preserved hole 43 through the main stent 42, D: removing the membrane 41 on the sub-stent 44 so that the sub-stent 44 can extend in the branch artery and secured at the preserved hole 43 on the main stent 42, E: repeating steps C and D to complete the other sub-stents 44 in other branch arteries.

However, the preserved holes (for the stents) are usually located at or near the outlets of the first arm artery, left common carotid artery and the left subclavian artery. After the main stent is located at a predetermined position, a metal wire penetrates the preserved hole on the main stent and secured another stent with appropriate size on the main stent in the vessel. Therefore, accurate intraoperative angiography would be necessary in clinical practice. Although it is minimally invasive, it is difficult and complicated in operation.

Therefore, there remains a need for a new and improved thoracic aortic stent structure to overcome the abovementioned issues to effectively to reconstruct vascular pathway.

### SUMMARY OF THE INVENTION

Conventionally, the preserved holes (for the thoracic aortic stents) are usually located at or near the outlets of the first arm artery, left common carotid artery and the left subclavian artery. After the main stent is located at a predetermined position, a metal wire penetrates the preserved hole on the main stent and secured another stent with appropriate size on the main stent in the vessel. Therefore, accurate intraoperative angiography would be necessary in clinical practice. Although it is minimally invasive, it is difficult and complicated in operation.

The present invention provides a thoracic aortic stent structure, including: (1) a main stent with a diameter similar to that of a human thoracic aorta, having at least one sub-stent formed at said main stent as one piece and diameter of the at least one sub-stent is corresponding to the first arm artery, the left common carotid artery and the left subclavian artery; (2) a membrane, covering outer surfaces of the main stent and the at least one sub-stent and forming at least one, side-opening at free end thereof, wherein the membrane compresses the main stent and the at least one sub-stent to narrow diameters thereof, and upon removing the membrane, the main stent and said at least one sub-stent are extended to reconstruct a vascular pathway.

### BRIEF DESCRIPTION OF THE DRAWINGS

FIG. 1A illustrates a three-dimensional view of one embodiment of the present invention.

FIG. 1B illustrates a three-dimensional view of another embodiment of the present invention.

FIG. 1C illustrates a three-dimensional view of a further embodiment of the present invention.

FIG. 2 is a schematic view of a first step for setting up a stent in the present invention.

FIG. 2A illustrates a partially enlarged view of a membrane used in one embodiment of the present invention.

FIG. 3 is a schematic view of a second step for setting up a stent in the present invention.

FIG. 4 is a schematic view of completion in setting up a stent in the present invention.

FIG. 5A is a schematic view of a first step for conventionally setting up a thoracic aortic stent.

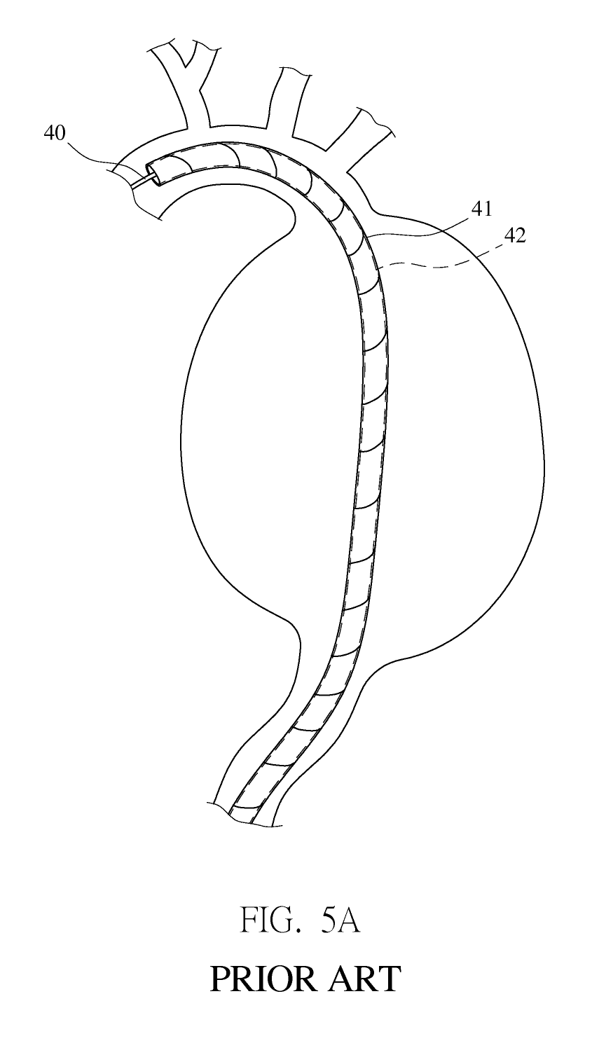

FIG. 5B is a schematic view of a second step for conventionally setting up a thoracic aortic stent.

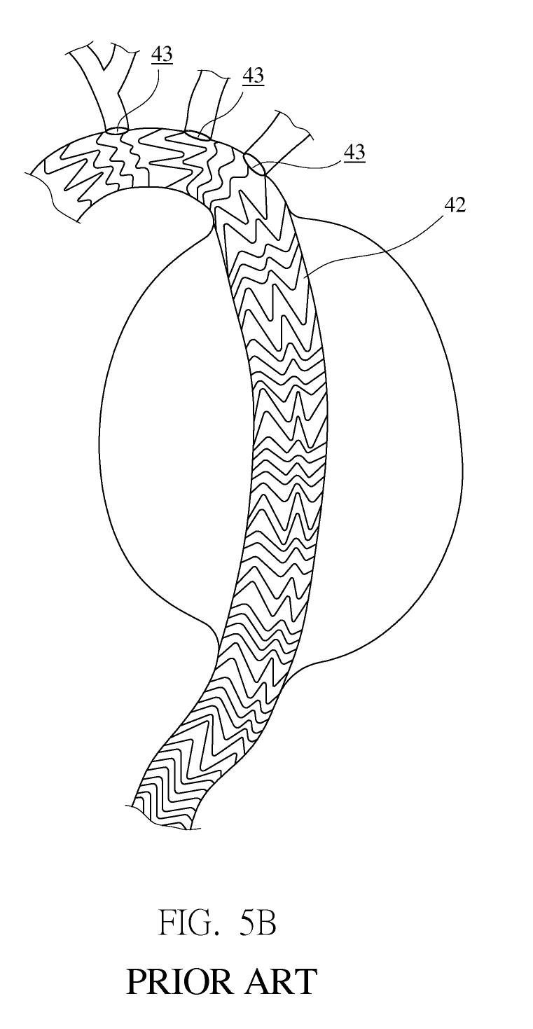

FIG. 5C is a schematic view of a third step for conventionally setting up a thoracic aortic stent.

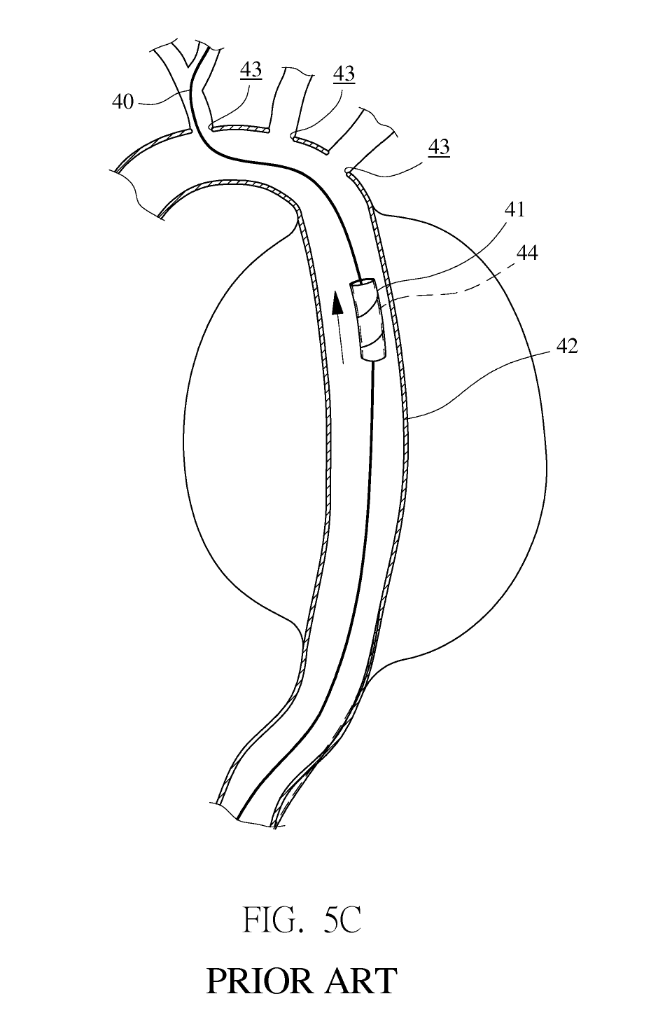

FIG. 5D is a schematic view of a fourth step for conventionally setting up a thoracic aortic stent.

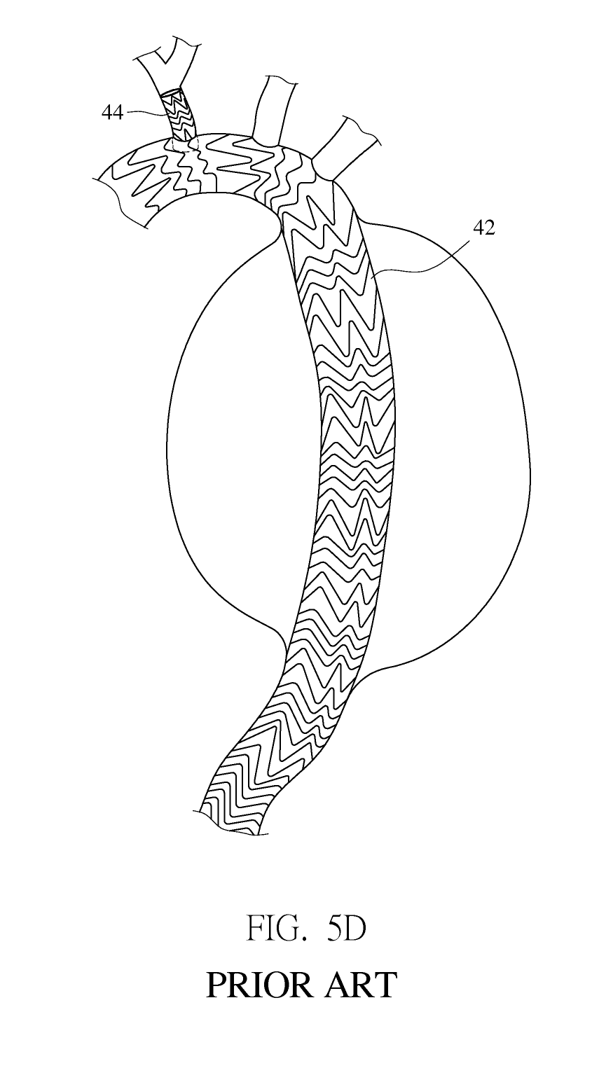

FIG. 5E is a schematic view of completion in conventionally setting up a thoracic aortic stent.

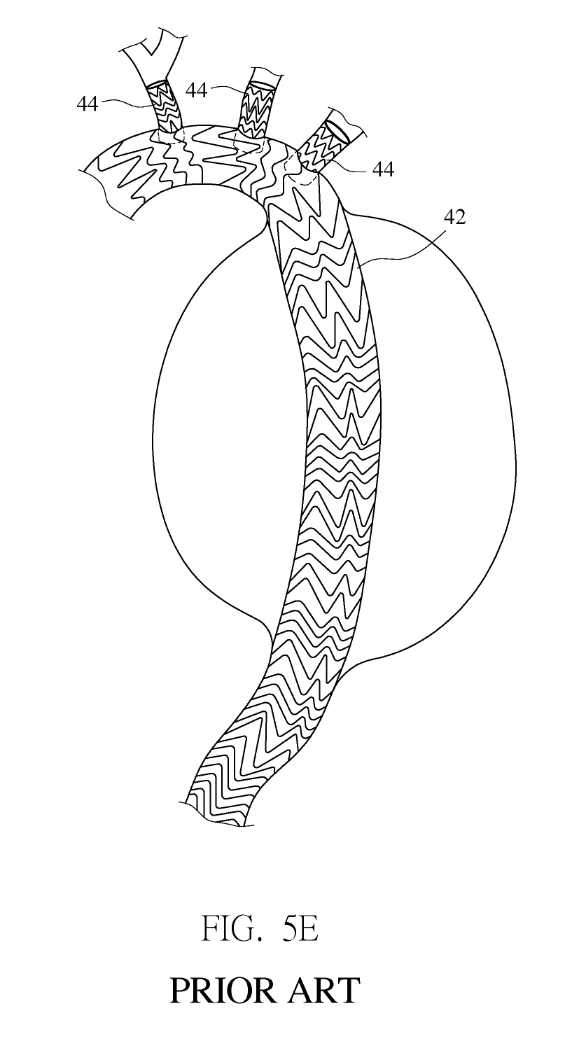

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications which might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

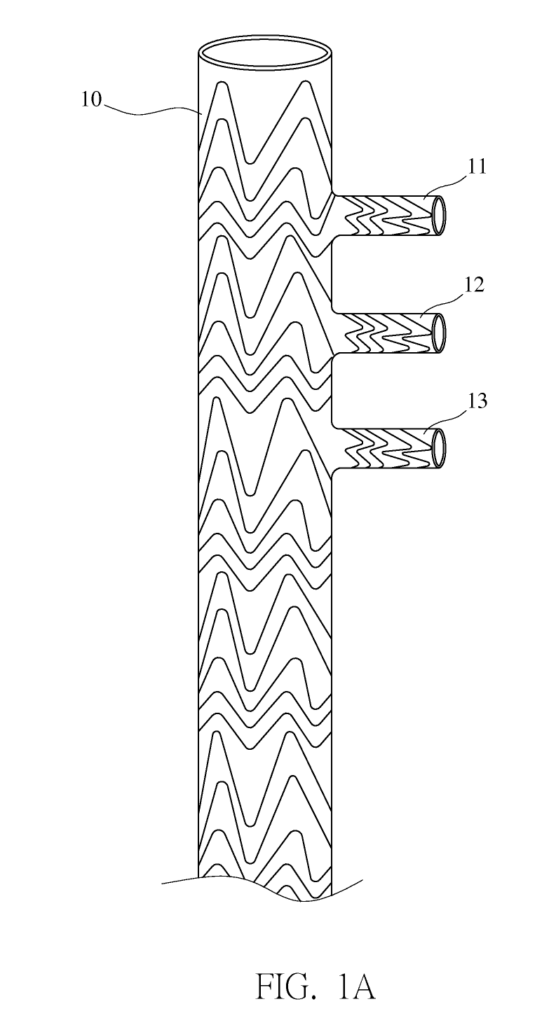

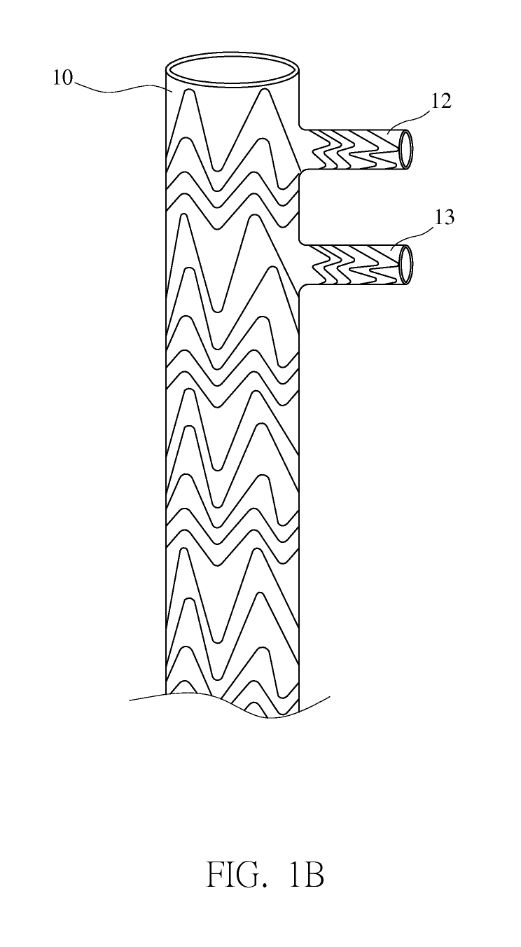

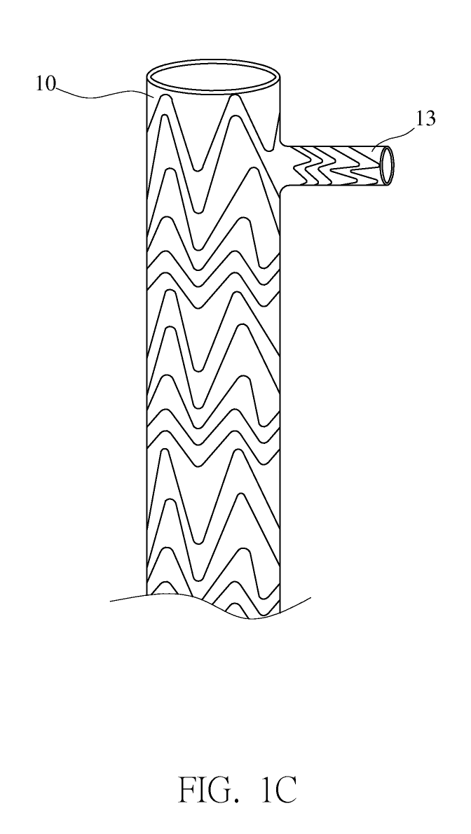

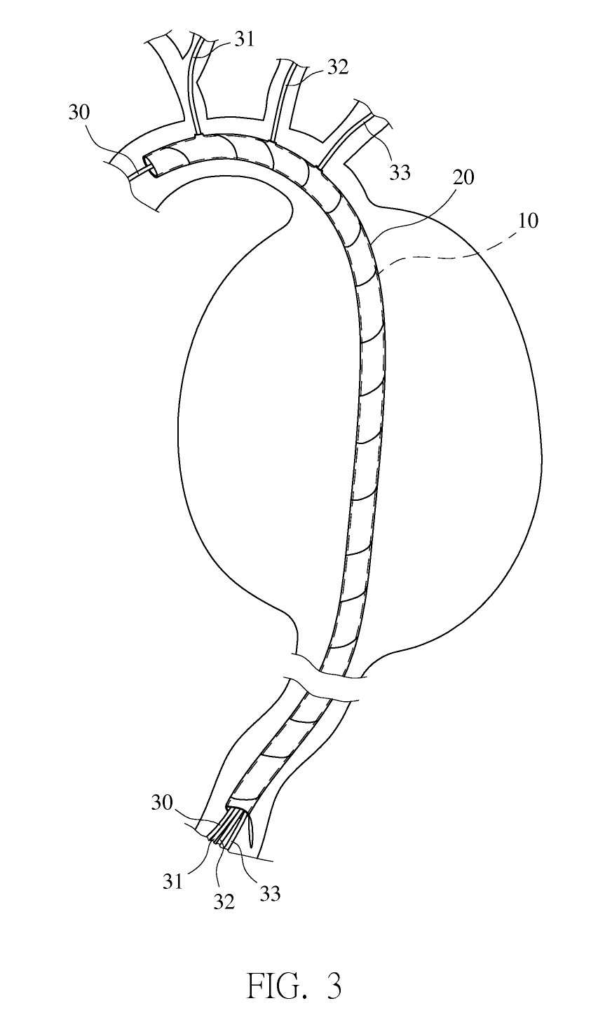

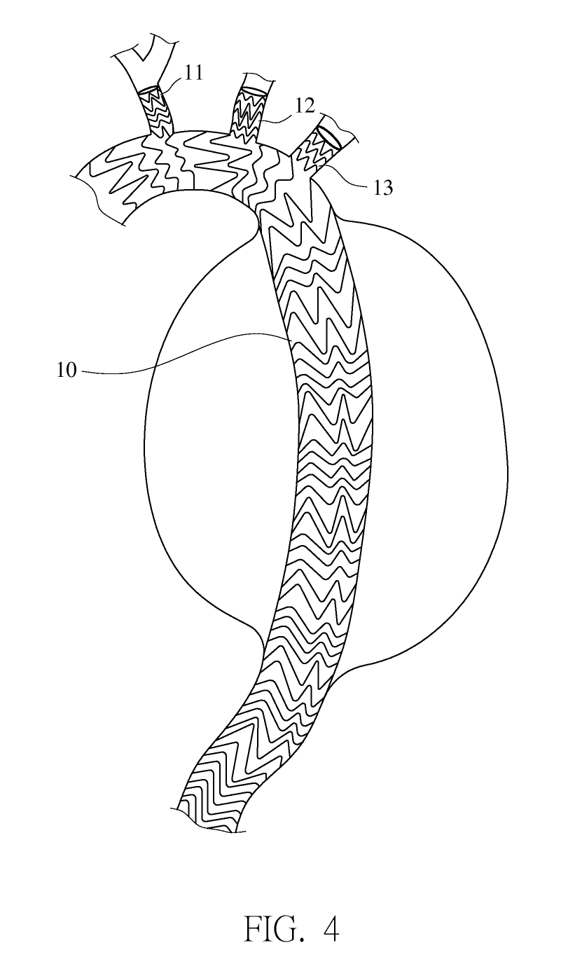

Referring to FIGS. 1 to 4, this invention is adapted to provide a thoracic aortic stent structure, including:
a main stent 10, diameter of the main stent 10 is similar to that of a human thoracic aorta, and corresponding to the human thoracic aorta, three sub-stents 11, 12, 13 are placed at the first arm artery, the left common carotid artery and the left subclavian artery. The diameter of each of the sub-stents 11, 12, 13 is similar to the first arm artery, left common carotid artery and the left subclavian artery, respectively, and each sub-stents forms at the main stent 10 as one piece;
a membrane 20, the membrane is formed at an outer surface of the main stent 10 and the three sub-stents 11, 12 and 13, and corresponding to the three sub-stents 11, 12 and 13, three side-openings 21, 22 and 23 are formed at free ends, wherein the membrane 20 compresses the main stent 10 and the three sub-stents 11, 12 and 13 to narrow the diameters thereof, and when the membrane 20 is removed, the main stent 10 and the three sub-stents 11, 12 and 13 are extended to reconstruct a vascular pathway;
wherein it may be unnecessary to place the sub-stents at each of the first arm artery, the left common carotid artery and the left subclavian artery according to the tumor's position of the patient, and one or two sub-stents may be sufficient.

Referring to FIGS. 1-B and 2, when only two sub-stents are necessary, two sub-stents 12 and 13 are placed at the left common carotid artery and the left subclavian artery corresponding to the main stent 10 at the human thoracic aorta. The diameters of the two sub-stents 12 and 13 are similar to that of the left common carotid artery and the left subclavian artery, and the two sub-stents 12 and 13 are formed at the main stent 10 as one piece. The membrane 20 is formed at an outer surface of the main stent 10 and the two sub-stents 12 and 13, and corresponding to the two sub-stents 12 and 13, two side-openings 22 and 23 are formed at free ends.

Referring to FIGS. 1-C and 2, when only one sub-stent is necessary, one sub-stent 13 is placed at the left subclavian artery corresponding to the main stent 10 at the human thoracic aorta. The diameter of the sub-stent 13 is similar to that of the left subclavian artery, and the sub-stent 13 is formed at the main stent 10 as one piece. The membrane 20 is formed at an outer surface of the main stent 10 and the sub-stent 13, and corresponding to the sub-stent 13, a side-opening 23 is formed at a free end,
wherein when the stent is extended but cannot meet the sealing effect as expected in clinic, an additional metal stent with appropriate size can be attached to the original stent through a metal wire, and a stent with appropriate size can be placed into the first arm artery, the left common carotid artery and the left subclavian artery.

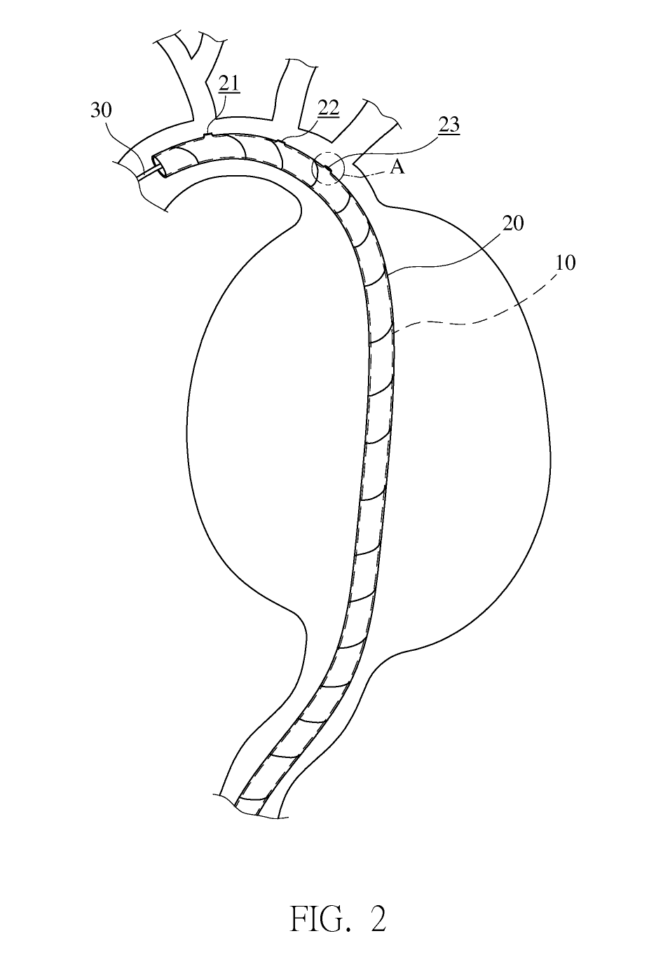

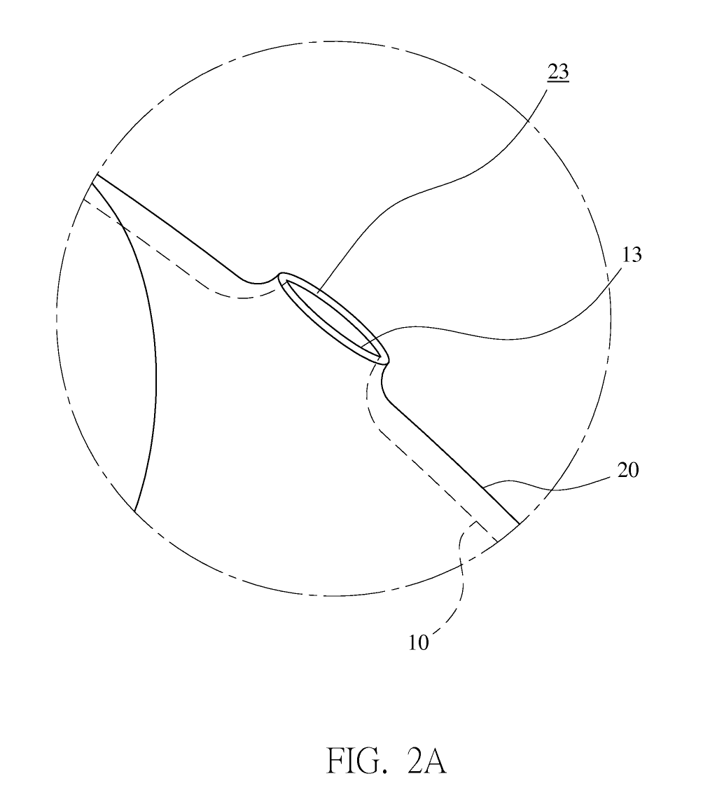

Referring to FIGS. 2 to 4, considering an example of a thoracic aortic stent with three sub-stents, the aortic stent includes a main stent 10 and three sub-stents 11, 12 and 13. Therefore, four metal wires 30, 31, 32 and 33 can be put inside the main stent 10 with the membrane 20, and the metal wire 30 can be put inside the femoral artery on both sides, such that the metal wire 30 can be hooked to the ascending aorta. Also, the main stent 10 with the membrane 20 moves along the metal wire 30 to the thoracic aorta. When reaching the predetermined position, the metal wires 31, 32 and 33 are hooked to the first arm artery, the left common carotid artery and the left subclavian artery through the side-opening 21, 22, and 23. Upon removing the membrane 20, the sub-stents 11, 12 and 13 in the first arm artery, the left common carotid artery and the left subclavian artery are in order opened, until the main stent 10 is extended. Therefore, the vascular pathway is reconstructed, and the process is easier and more convenient to operate, which can also shorten the time, for surgery.

Having described the invention by the description and illustrations above, it should be understood that these are exemplary of the invention and are not to be considered as limiting. Accordingly, the invention is not to be considered as limited by the foregoing description, but includes any equivalents.

What is claimed is:

1. A thoracic aortic stent structure, comprising:
a main stent with a diameter similar to that of a human thoracic aorta, having at least one sub-stent which is formed at said main stent as one piece, and at least one sub-stent having a diameter which is similar to that of a corresponding left subclavian artery; and
a membrane, covering the main stent and said at least one sub-stent and forming at least one side-opening at a free end of the at least one sub-stent, wherein the membrane compresses the main stent and said at least one sub-stent to narrow diameters thereof, and upon removing the, membrane, the main stent and said at least one sub-stent are extended to reconstruct a vascular pathway.

2. The thoracic aortic stent structure of claim 1, wherein the main stent as to the human thoracic aorta has two sub-stents corresponding to said left subclavian artery and left common carotid artery, and the diameters of the two sub-stents are similar to, that of the left subclavian artery and left common carotid artery, and the two sub-stents are formed at the main stent as one piece having two side-openings at free ends.

3. The thoracic aortic stent structure of claim 1, wherein the main stent as to the human thoracic aorta has three sub-stents corresponding to the first arm artery, the left common carotid artery and the left subclavian artery, and the diameters of the three sub-stents are similar to that of the first arm artery, the left common carotid artery and the left subclavian artery, and the three sub-stents are formed at the main stent as one piece having three side openings at free ends.
